(19) United States
(12) Patent Application Publication
(10) Pub. No.: US 2009/0138031 A1
(43) Pub. Date: May 28, 2009

(54) Thrombectomy catheter with a helical cutter

(76) Inventor: Vladimir B. Tsukernik, West Roxbury, MA (US)

Correspondence Address: VLADIMIR B. TSUKERNIK, 206 Mt.. Vernon St.., West Roxbury, MA, 02132, US

(21) Appl. No.: 12/292,730

(22) Filed: Nov. 14, 2008

Related U.S. Application Data
(60) Provisional application No. 61/004,130, filed on Nov. 24, 2007.

(51) Int. Cl. A61B 17/22

(52) U.S. Cl. 606/159

(57) ABSTRACT

A thrombectomy catheter including two major members: a cutter of the helically wounded structure and also a cutter of the tubular shape which is located coaxially with the first one. The outside diameter of the helical cutter fits inside diameter of tubular one with a small gap. Thus they cold move independently. Each of them is equipped with the cutting edges on their distal area. Due to this feature the catheter is able to cut off the major portion of the obstacle from a vessel without its fragmentation and safe removing it off the vessel. The catheter is also providing blood perfusion during the surgery as well as provides opportunity for the monitoring of the operation by means of ultrasound, visual etc. devices during the operation. The original flexible design of the helical cutter also prevents the damage of the vessel.

The present invention generally relates to thrombectomy or atherectomy devices and particularly to thrombectomy catheter devices.

A variety of techniques and instruments have been developed to remove obstructive material from arteries or other body passageways or to repair the ones.

A frequent objective of such techniques and instruments is the removal of atherosclerotic plaques in the patient's arteries. The buildup of these initially fatty deposits characterizes atherosclerosis. It may be referred to as stenotic lesions or plaques while the blocking material may be referred to as stenotic material.

Several kinds of thrombectomy devices have been developed for attempting to remove some or all of such stenotic material. In one type of device, such as that shown in U.S. Pat. No. 5,092,873 a cylindrical housing, carried at the distal end of a catheter, has a portion of its side-wall cut out to form a window into which the stenotic lesion can protrude when the device is positions next to the plaque. A thrombectomy blade, disposed within the housing, advancing the length of the housing to cut the portion of the plaque that extend into the housing cavity. While such device provides for directional control in selection of tissue to be extracted the length, rigidity and outside diameter of the cylindrical housing limits maneuverability and therefore also limit the utility of the device.

Another approach, which solves some of the problems related to removal of plaques in narrow and tortuous passageways, involves the use of an offset-agitator. Example of such device is illustrated in U.S. Pat. No. 6,758,851. In this device the offset-agitator, which is a flexible helical spring, exposes its distal end beyond the distal face of the flexible tubing. The motor rotates the offset-agitator, which extended distal end, fragments the plug and conveys the fragments out of the vessel by means of the negative pressure and conveyor-shaft inside the flexible tubing. While this device could destroy plugs inside vessels it also cold seriously damage the elastic tissue of the vessel as well. Also the device doesn't provide any guarantee in removing all fragments, and small particles from the area of cutting thus contaminating the blood.

None of these devices approximates the design of the device described below.

### SUMMARY OF THE INVENTION

Current pharmacological, surgical or trans-catheter procedures for opening clogged vessels can be time consuming, traumatic and expensive as well as most of them remain myriad of small particles of material of destroyed plugs, circulating in the patient's body.

Objects of the present invention are:

To provide a flexible apparatus that can be inserted into patient's blood vessel thru a small puncture wound, be navigated to an obstruction like thrombus.

To remove the all or significant portion of this obstacle without its fragmentizing.

Do not damage the wall of vessels.

To provide blood perfusion in the area of operation.

To provide opportunity for visual, ultrasound, etc. direct monitoring of the surgery during the process.

There is provided in accordance with the present invention a thrombectomy catheter adapted to access remote obstacle in the vascular system.

The catheter comprises an elongate flexible tubular body (referred as “cutter tubing”) having a sufficiently small outside diameter to reach the smallest vessel's interior and at the same time big enough to create only a small gap between itself and the vessel's inside diameter (ID). The tubular body also has sufficient kink resistance, pushability and ability to transmit sufficient torque. In accordance with the invention the distal end of the “cutter tubing” is permanently connected to the proximal end of the metal cutter of the tubular shape (referred as a “tubular cutter”) with the cutting edges tapered inwardly on its distal side. The proximal end of the “cutter tubing” connected to the manual control with the ability of longitudinal and rotational movement

The configuration of the device also includes another flexible tubular body (referred as a “screw tubing”) located coaxially and inside the “cutter tubing” and has sufficient kink resistance, pushability and ability to transmit sufficient torque. In accordance with the present invention the distal end of the “screw tubing” permanently connected to the proximal end of the cutter having a cylindrical helical shape, reminding corkscrew shape. (Referred as a “corkscrew cutter”).

The outside diameter (OD) of the “corkscrew cutter” fits the inside diameter of the “tubular cutter” with the smallest gap between them and also fits “cutter tubing's” ID. The distal end of the “corkscrew cutter” equipped with the “cutting tip” that provides the penetration of the “corkscrew cutter” into the stenotic lesion material and has rounded edges. The proximal end of the “screw tubing” connected to the manual control with the ability of longitudinal and rotational movement.

The low risk of vessel's wall damage is provided by very good alignment of the “corkscrew cutter” and “tubular cutter” with the lumen of the vessel because the gap between the vessel ID and “tubular cutter” OD as well as “cutter tubing” is very small. Another version of the invention describes the “corkscrew cutter ” as the system of at least of two segments of helix connected by joints in some specific way described below. This significantly improve the flexibility of the “corkscrew cutter” and its safety. In addition to this the rounded edges of the “cutting tip” also improve the safety of the catheter.

The lumen of the “screw tubing” and the room inside the “corkscrew cutter” could be used for inserting borescope, ultrasound catheter or any other type of devices for the direct monitoring of the surgery.

In accordance with a further aspect of the present invention there is provided a method of removing material from a vessel. The method comprises the steps of providing at least two flexible tubular bodies coaxially located one inside another with the ability of free movement relatively each other, having a proximal ends and a distal ends, two types of cutters attached to the distal end of each tubular bodies and also manual control on the proximal ends of these tubular bodies. The device is advanced transluminally to the destination area until it reaches stenotic lesions. From this point “corkscrew cutter” start the rotational movement with the corresponding longitudinal advance, penetrating the material of the lesion. The “tubular cutter” just follows “corkscrew cutter” performing rotational and longitudinal movement sliding along outside surface of the “corkscrew cutter” shearing the material of lesion by its front edge thus material of the lesion remains locked inside the “corkscrew cutter” area and could be easy removed from the vessel together with the catheter.

Further features and advantages of the present invention will become apparent to those of skill in the art in view of the disclosure herein, when considered together with the attached drawings and claims.

### BRIEF DESCRIPTION OF THE FIGURES

FIG. 1 is a schematic view of a device embodying the present invention.

FIG. 2 is a partially sectioned side view of the distal end of the device of FIG. 1 showing an embodiment of distal sleeve assembly.

FIG. 3 is a partially sectioned view of the distal end of the device of FIG. 1 showing only cutters and tubings that attached to them.

FIG. 4 is a view of the distal and proximal ends of the device of FIG. 1

FIG. 5 is a view of a tubular cutter.

FIGS. 6a, 6b, 6c, 6d, 6e are the different embodiments of a view “A” of FIG. 3

FIGS. 7a, 7b, 7c are different embodiments of a section “B-B” of FIG. 3

FIG. 8 is a partially sectioned view of a distal end of the device of FIG. 1 located in some proximity to the lesion.

FIG. 9 is a partially sectioned view of a distal end of the device of FIG. 1 in the position about to start cutting.

FIG. 10 is a partially sectioned view of a distal end of the device of FIG. 1 during the process of the lesion penetration.

FIG. 11 is a partially sectioned view of a distal end of the device of FIG. 1 after the screw cutter penetrated completely through the stenotic lesion.

FIG. 12 is a partially sectioned view of a distal end of the device of FIG. 1 during the process of removing part of the lesion from the vessel.

FIGS. 13, 14 and 15 is a partially sectioned view of the distal end of the device of FIG. 1 with the different embodiments of the helical cutter.

### DETAILED DESCRIPTION OF THE PREFERRED EMBODIMENT

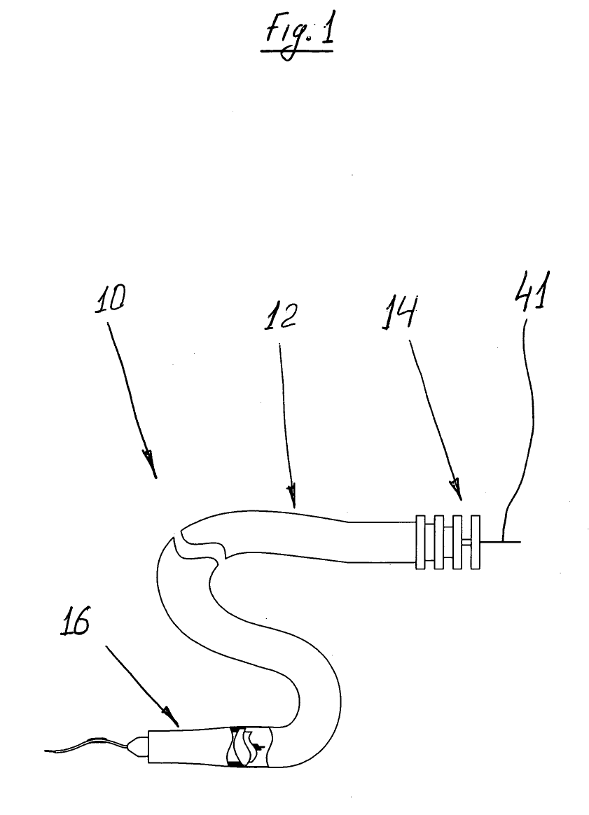

With reference initially to FIG. 1, a surgical instrument, indicated generally by reference numeral 10 comprises an elongate flexible tubular body 12, having a proximal end 14 and a distal end 16, as well as guide wire 41. A control 18 is preferably provided at or near the proximal end 14 of the tubular body 12 for permitting manipulation of the instrument 10.

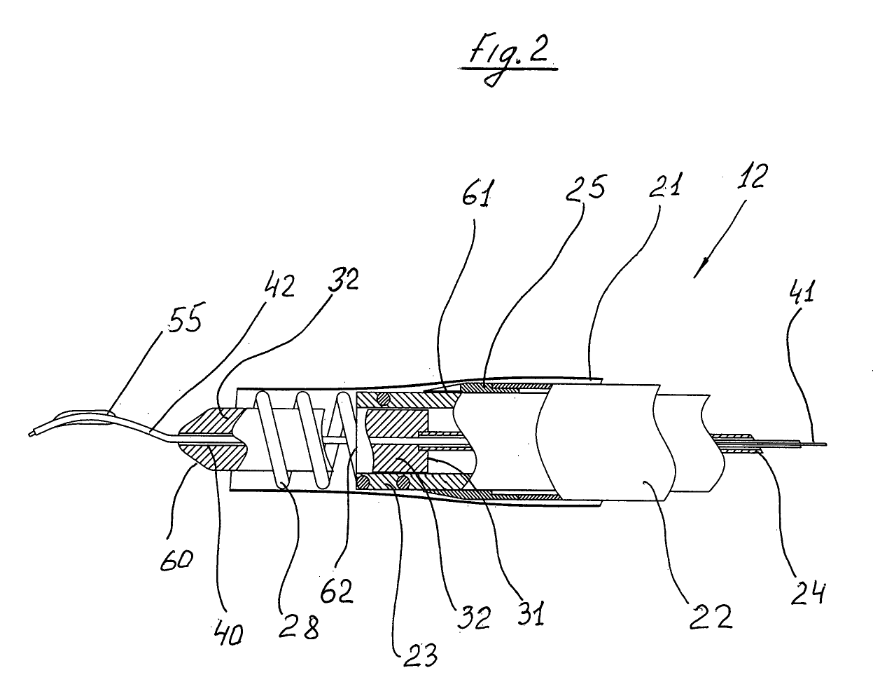

With reference now to particularly sectioned view of FIG. 2 the tubular body 12 comprises of four tubing coaxially located one inside the others.

The “outside tubing” 21 which represents the thin wall extrusion (wall thickness ˜0.0005 . . . 001″) comprises the “cutter tubing” 22. Inside of the cutter tubing 22 you could find “screw tubing” 23 which also comprises central tubing 24.

With the reference to the FIG. 3 the proximal area 26 of the tubular cutter 25 is permanently connected to the distal portion of the tubing 22 by any available means which aren't discussed. The corkscrew cutter 25 and tubing 22 are coaxial. Also the corkscrew cutter 28 with its proximal area 29 IIIbI permanently connected to the distal portion 30 of the tubing 23. Parts 28 and 25 are also coaxial.

There is also some small gap 76 between tubing 23 OD and tubing 22 ID to provide the blood perfusion during the surgery.

With the reference again to the FIG. 2 the central tubing 24 located in the lumen of tubing 23 and distal portion of tubing 24 is permanently connected to the proximal portion 31 of the insert 32. The insert 32 is coaxial with the corkscrew cutter 28. The insert 32 has also the central lumen 40 to accept a guide wire 41 and possible balloon catheter 42.

All above mentioned tubing could be independently rotated and moved longitudinally.

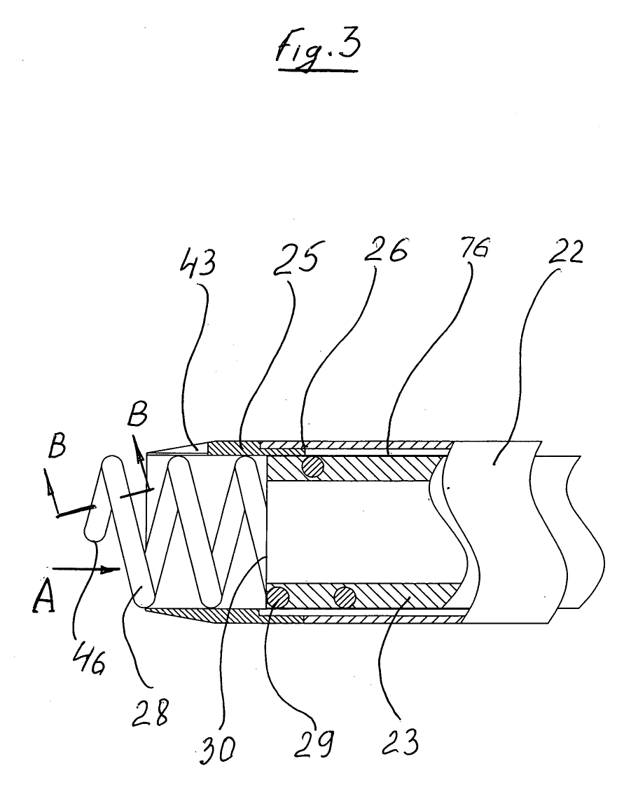

With the reference now to FIG. 4 the illustrated proximal area of device 10 of FIG. 1 schematically represents the control area of the device 10. The knobs 33, 34, 35, 36 permanently connected to the tubings 21, 22, 23 and 24 correspondingly to provide necessary control of the movement of above mentioned tubings and thus working pieces on the distal ends of these tubings.

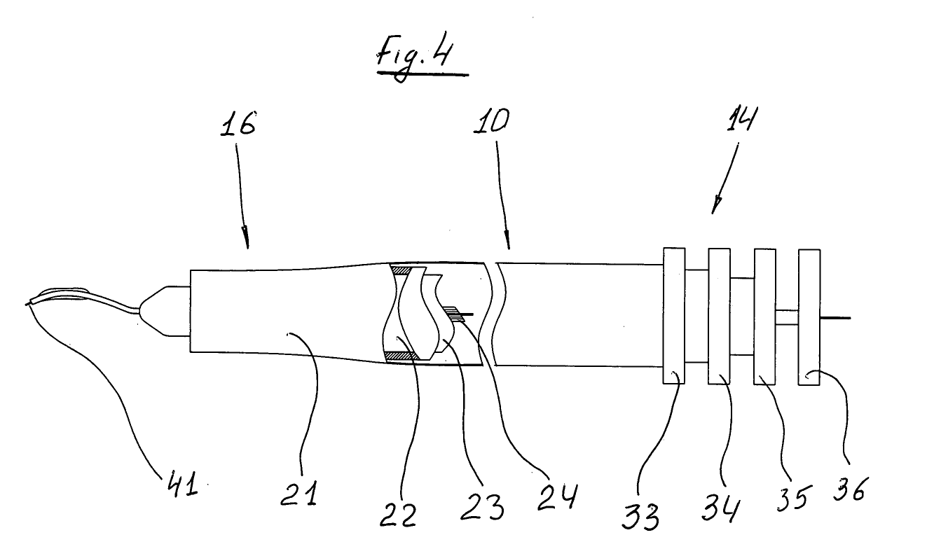

With the reference to FIG. 3 tubular cutter 25 represents the tubular body with the outside diameter (OD) equal or smaller than vessel's inside diameter (ID). Also ID of cutter 25 is ˜0.001 . . . 003 bigger than OD of tubing 23 and corkscrew cutter 28. The distal end of the cutter 25 is its cutting edge 43 and the shape of the edge 43 could be different as illustrated on FIG. 5.

With the reference to FIG. 5 the shape of the cutting edge 43 could be curved FIG. 5a or straight FIG. 5b. The length 44 of the cutter is not less than the distance covered by two coils of the corkscrew cutter 28.

With the reference to FIG. 3 the corkscrew cutter 28 has a shape of helix or to say “corkscrew”. The proximal portion 29 of it adjusted to the specific attachment to the distal portion of the tubing 23. The distal portion of the cutter 28 is equipped with a cutting tip 46.

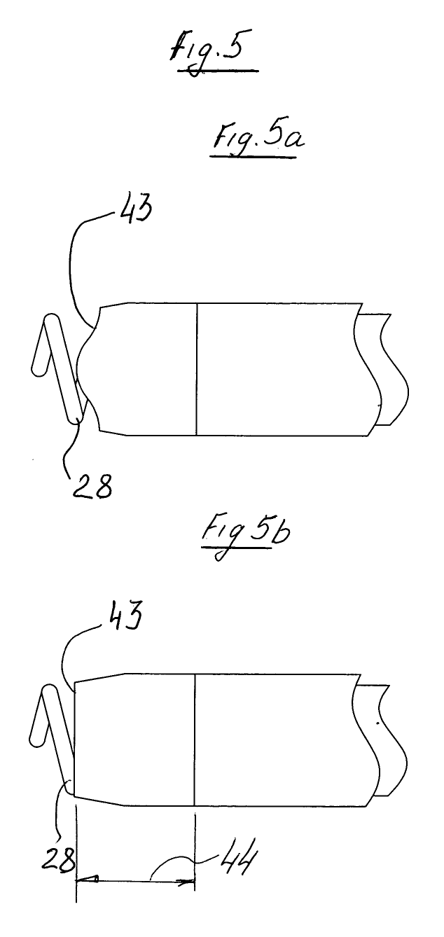

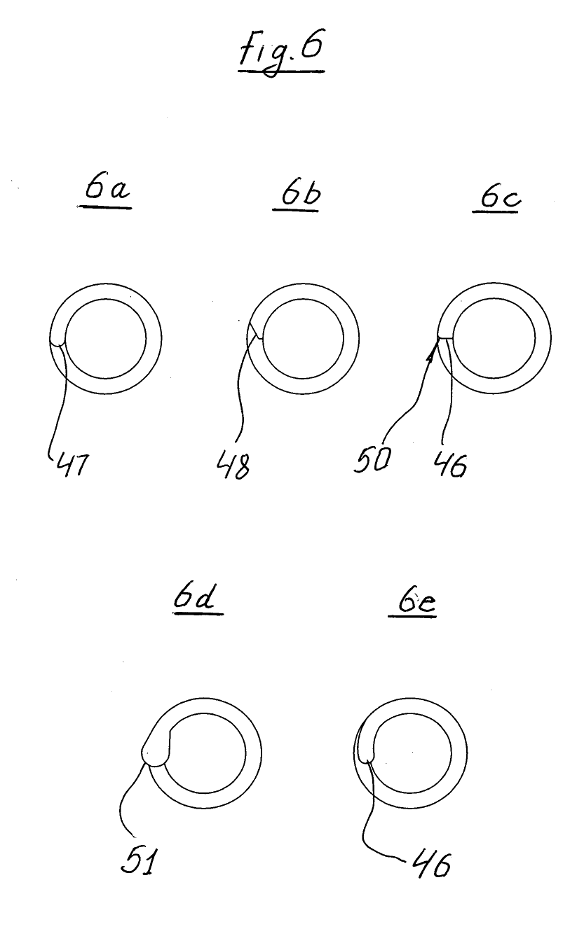

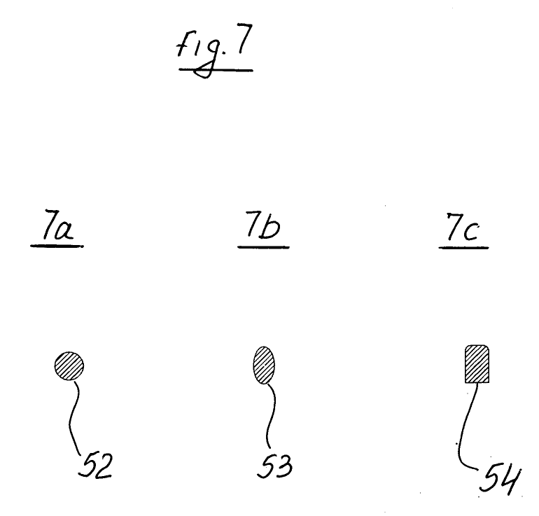

FIGS. 6a, 6b, 6c, 6d, 6e, 6f are illustrating different shapes of tip 46. FIG. 6a demonstrates hemispherical shape 47 of the tip 46. FIG. 6b represents chamfer shape 48 of the same tip 46. And also FIG. 6c presents the square cut 48 with a fillet 50.Another embodiment of this solution could be seen on FIG. 6d. With the bulb 51 on the tip 46. FIG. 6e illustrates the tip 46 slightly bended inwardly.

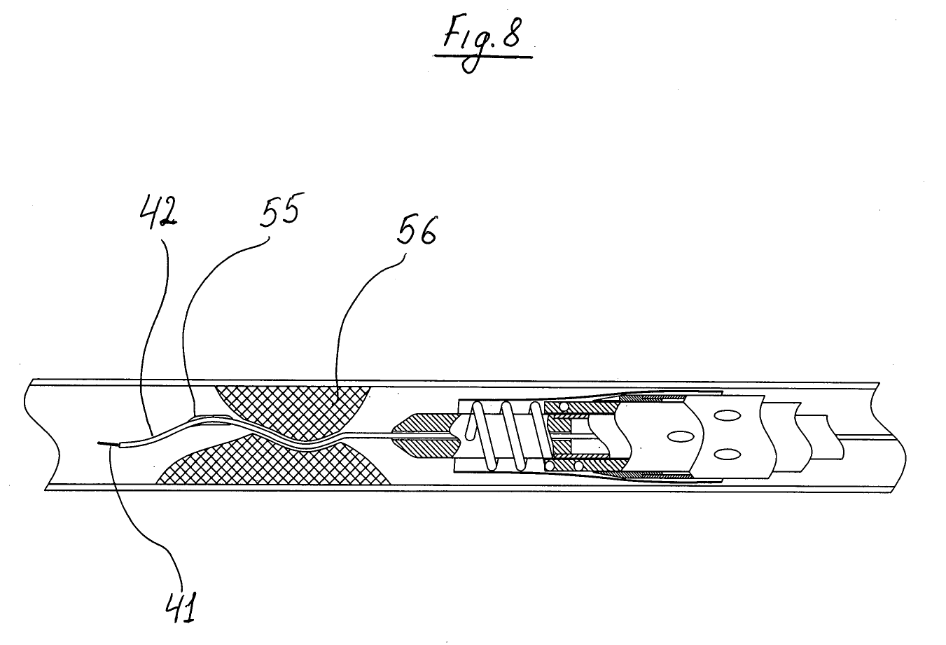

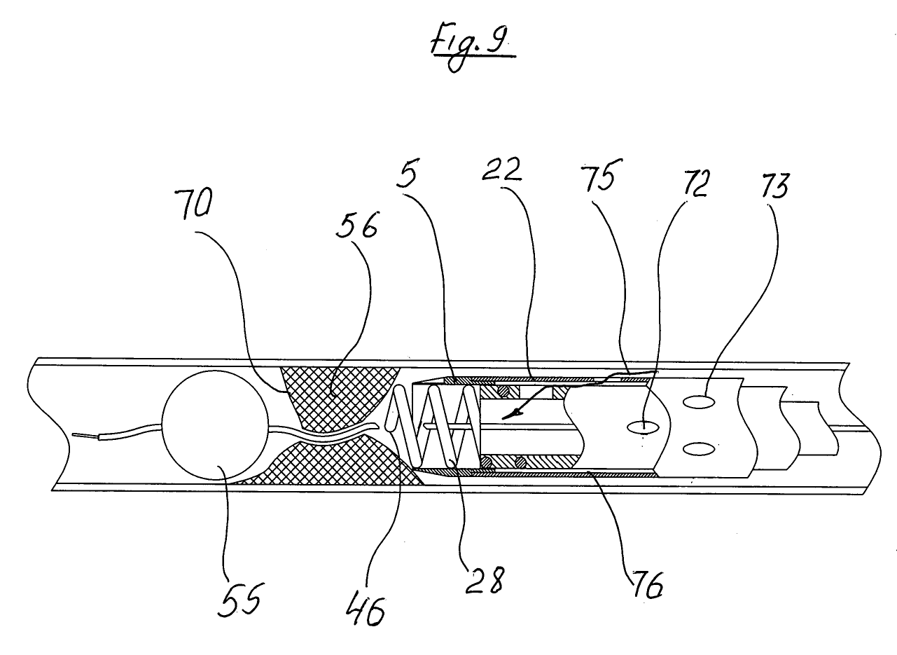

Also different profiles of the wire of the corkscrew cutter 28 are represented on the FIGS. 7a, 7b, 7c. Circular shape 52, oval shape 53, and rectangular shape 54 could be seen on these FIGs.

Referring again to FIG. 2 insert 32 is positioned coaxially and inside of the cutter 28 and tube 23. OD of the insert 32 fits the ID of the cutter 28 and tubing 23 with a gap (˜0.002″) which allows moving it freely inside the lumen.

To be introduced into the vessel the catheter is to be in configuration shown on FIG. 2. Specifically: the distal tip 60 of insert 32 is to be advanced relatively to the tip 46 of the corkscrew cutter 28, preventing the damage of the vessel's wall by tip 46 during the advancing of the catheter inside a vessel. The distal area 61 of the tubular cutter 25 is positioned behind the proximal area 62 of the cutter 28 on the tubing 23. Balloon 55 of a “balloon catheter” 42 extends beyond the distal tip 60 of the insert 32. On FIG. 4 the sleeve 21 covers all area of the device 10 from its distal end 16 thru the knob 33 on proximal end 14.Tubing 21 provides additional protection of the vessel's wall.

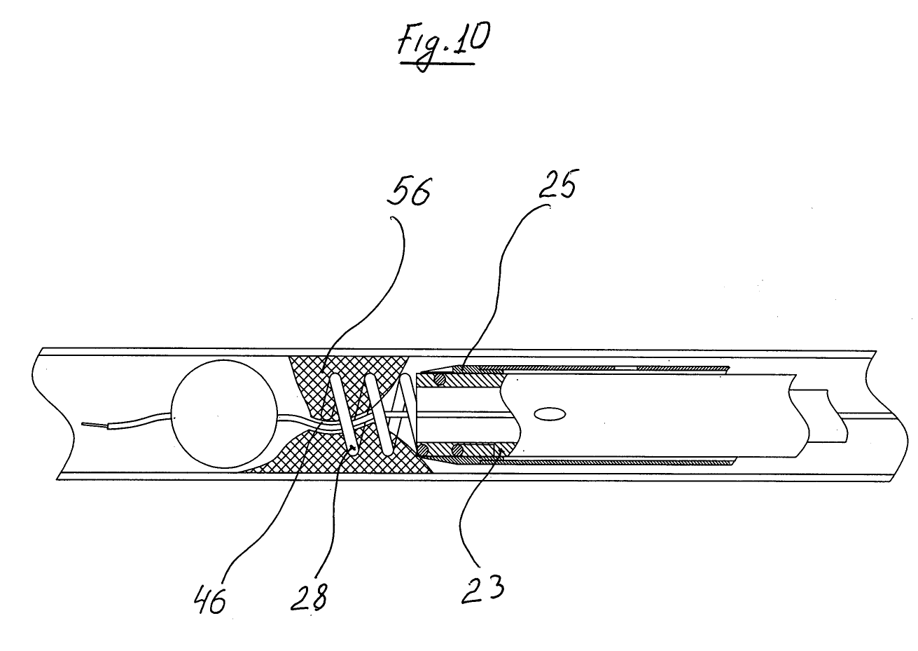

In a presently preferred method of use on FIG. 8, a guide wire 41 is first percutaneously introduced and transluminally advanced in accordance with well known techniques to the obstruction to be cleared. The device 10 is then introduced by placing the distal end 16 of the flexible tubular body 12 on the guide wire 41, and advancing the flexible tubular body 12 along the guide wire 41 through the vessel to the treatment side. Balloon 55 of balloon catheter 42 advanced the lesion area and placed behind the lesion. The device stops at the close proximity to the stenotic lesion 56.

FIG. 9 represents the configuration of the distal end 16 before the beginning of the cutting a lesion 56. Outside sleeve 21 is pulled back from the distal end 16 by means of knob 33. Insert 32 is removed from the area of distal end 16 by means of knob 36. Tubular cutter 25 with tubing 22 advanced by means of knob 34 to the close proximity of tip 46 to provide necessary support for the corkscrew cutter 28. Balloon 55 is inflated and support the distal area 70 of the lesion preventing it from dislodging The blood perfusion is provided by means of holes 72, 73 as well as gap 76. The arrow 75 shows the blood stream.

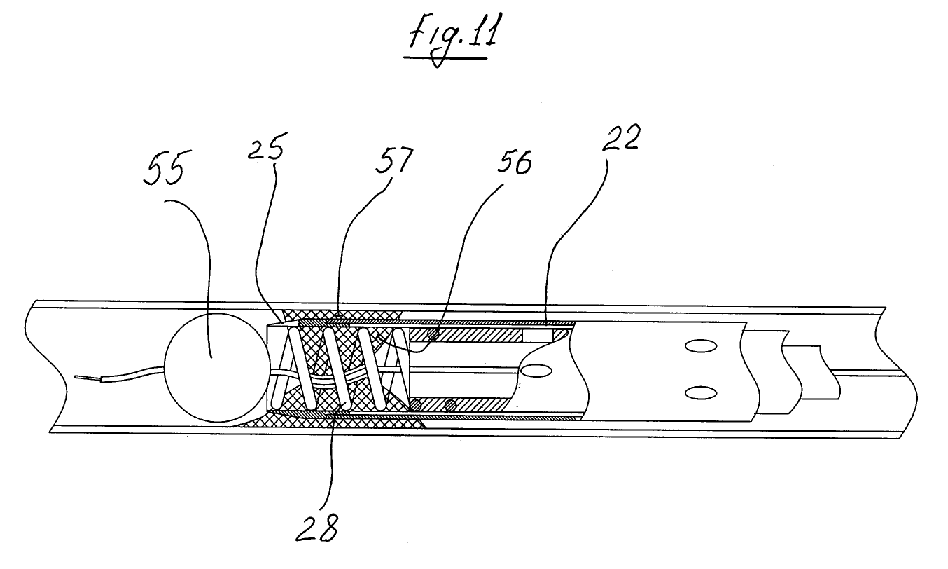

FIG. 10 illustrates the cutting process. The corkscrew cutter 28 by means of tubing 23 and knob 35 starts rotation with some longitudinal advance. At this moment tip 46 penetrates the lesion material 56, cutter 28 gets through the lesion 56 and tubing cutter 25 follows it.

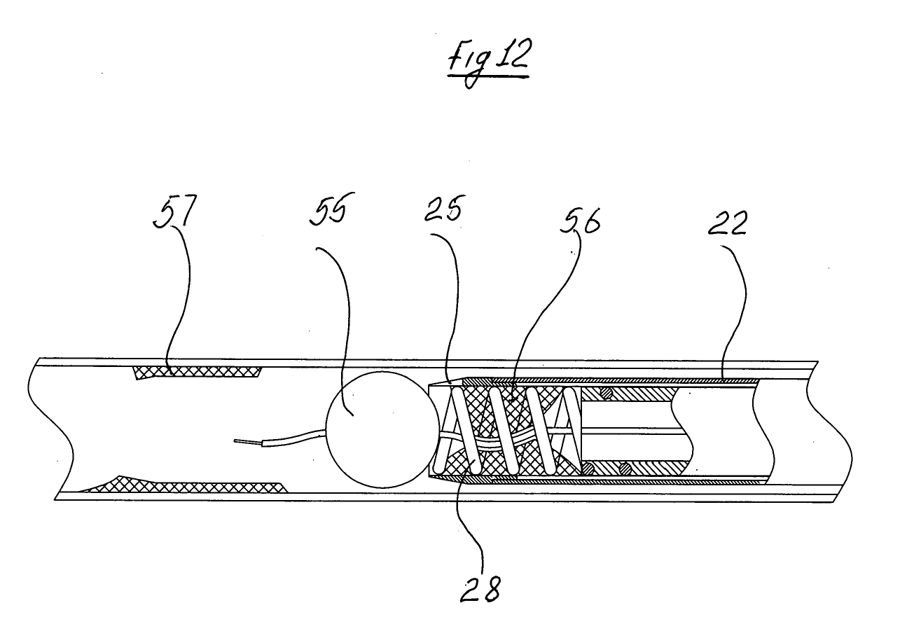

FIG. 11 represents the final steps of the cutting lesion. During this process the tubular cutter 25, operated by means of knob 34, follows the corkscrew cutter 28, sliding along its outside surface. This cuts off the removing lesion material 56 from the rest of it 57 located to the close proximity to the vessel's wall. By the end of the cutting process significant part of the stenotic plaque 56 is locked within area designated by tubular cutter 25 and tubing 22 outside and by inflated balloon 55 on the distal side. It could be removed from the vessel.

FIG. 12. demonstrates the final process removing of the catheter from the vessel with the portion of plug inside it. The removable portion of the lesion material 56 is locked within tubular cutter 25, tubing 22 and inflated balloon 55, 28 is a corkscrew cutter, 57 is a remaining part of the lesion material.

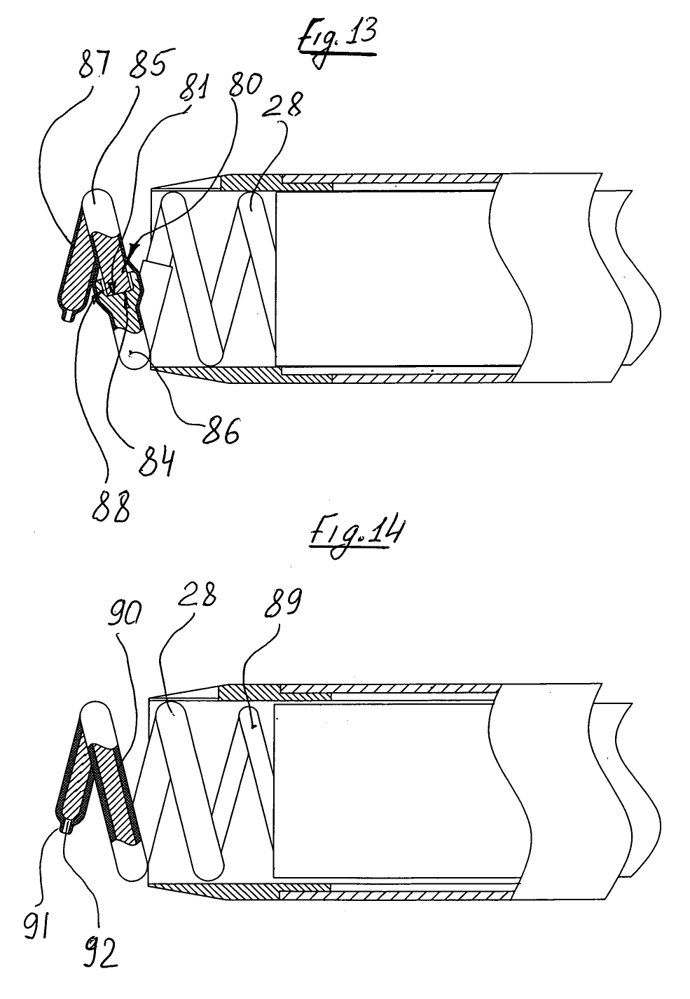

FIG. 13 represents another embodiment of the cutter 28. In this case the cutter consists of a few segments (2 parts are shown) 85 and 86 of the same outside diameter, pitch and shape. These parts are connected to each other by means of joint 80. Joint 80 is created by means of proximal face 81 of the wire of the segment 85 and by the distal face 84 of the extended diameter of the wire of the segment 86. The face 84 could have a shape of the cup 88 to contain the face 81 inside itself as it shown. Segments 85 and 86 are hold together by means of heat-shrink tubing 87

FIG. 14 represents another version of the cutter 28 which consists of the metal wire 89 of the helix shape covered by the plastic coating 90 or heat shrink tubing. The FIG. 14 as well as FIG. 13 represents the tip 91 of the helical cutter. The tip 91 is created by means of the heat-shrink tubing 90 which extend beyond the distal face 92 of the wire 89.

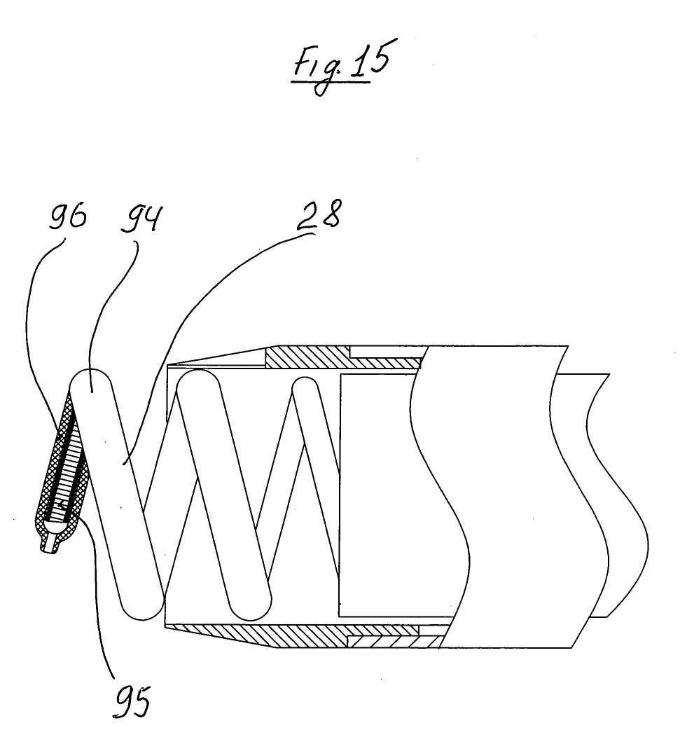

FIG. 15 illustrate another embodiment of the helical cutter 28. In this version the distal part 94 of the cutter 28 is created by the combination of the cylindrical extension spring 95 in the compressed state and by the heat shrink tubing 96 covering its outside surface.

While invention has been particularly shown and described with reference to preferred embodiments thereof, it will be understood by those skilled in the art that changes in form and detail may be made therein without departing from the spirit and scope of the invention.

What is claimed is:

1. A device for removing an obstruction located in a blood vessel, the device comprising:
(a) a tubular cutter, the tubular cutter being dimensioned for coaxial insertion into a blood vessel lumen, the tubular cutter comprising a proximal end, a distal end, and a lumen;
(b) a first length of flexible tubing, the first length of flexible tubing being coaxially disposed within the lumen of the tubular cutter, the first length of flexible tubing comprising a proximal end and a distal end; and
(c) a helical structure, the helical structure comprising a proximal end, a distal end and a longitudinal axis, the helical structure defining a lumen and being rotatable about its longitudinal axis, the proximal end of the helical structure being fixed to the distal end of the first length of flexible tubing, the distal end of the helical structure comprising a blunt tip.

2. The device as claimed in claim 1 wherein the tubular cutter is substantially uniform in inner diameter and wherein the distal end of the tubular cutter tapers inwardly in outer diameter.

3. The device as claimed in claim 2 wherein the distal end of the tubular cutter is shaped to include an externally-facing recess.

4. The device as claimed in claim 1 wherein the tubular cutter is a rigid body made of metal.

5. The device as claimed in claim 1 further comprising a second length of flexible tubing, the second length of flexible tubing comprising a proximal end and a distal end, the proximal end of the tubular cutter being fixed to the distal end of the second length of flexible tubing.

6. The device as claimed in claim 5 further comprising a third length of flexible tubing, the tubular cutter and the second length of flexible tubing being coaxially disposed within the third length of flexible tubing.

7. The device as claimed in claim 6 further comprising a tubular insert, the tubular insert comprising a proximal end, a distal end and a lumen, the tubular insert being coaxially disposed and slidably mounted within the first length of flexible tubing.

8. The device as claimed in claim 7 further comprising a fourth length of flexible tubing, the fourth length of flexible tubing comprising a proximal end, a distal end, and a lumen, the distal end of the fourth length of flexible tubing being fixedly mounted on the proximal end of the tubular insert, with the lumen of the fourth length of flexible tubing being aligned with the lumen of the tubular insert.

9. The device as claimed in claim 8 further comprising a guide wire, the guide wire being slidably mounted within the lumen of the fourth length of flexible tubing and the lumen of the tubular insert.

10. The device as claimed in claim 9 further comprising a balloon catheter, the balloon catheter being slidably mounted within the lumen of the fourth length of flexible tubing and the lumen of the tubular insert.

11. The device as claimed in claim 1 further comprising means for rotating and translationally moving the tubular cutter and the first length of flexible tubing independently of one another.

12. The device as claimed in claim 1 wherein the distal end of the tubular cutter has a curved shape.

13. The device as claimed in claim 1 wherein the distal end of the tubular cutter has a straight shape.

14. The device as claimed in claim 1 wherein the blunt tip of the helical structure has one of a hemispherical shape, a chamfer shape, a square-cut shape and a bulb shape.

15. The device as claimed in claim 1 wherein at least one of the first length of flexible tubing and the second length of flexible tubing has at least one transverse opening to permit blood perfusion.

16. The device as claimed in claim 1 wherein the helical structure comprises a plurality of segments joined together lengthwise with a length of heat-shrink tubing.

17. The device as claimed in claim 1 wherein the helical structure comprises a helical member covered by a length of heat-shrink tubing, the helical member having a distal end, the length of heat-shrink tubing having a distal end, the distal end of the heat-shrink tubing extending distally beyond the distal end of the helical member.

18. The device as claimed in claim 17 wherein the helical member comprises a cylindrical extension spring in a compressed state.

19. The device as claimed in claim 17 wherein the helical member comprises a wire.
